(19) United States
(12) Patent Application Publication
(10) Pub. No.: US 2005/0255135 A1
(43) Pub. Date: Nov. 17, 2005

(54) Cellulose microbeads and the matting of human skin therewith

(76) Inventor: Guillaume Cassin, Villebon Sur Yvette (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/121,920

(22) Filed: May 5, 2005

Related U.S. Application Data
(63) Continuation of application No. PCT/EP03/11646, filed on Oct. 20, 2003.
(60) Provisional application No. 60/429,342, filed on Nov. 27, 2002.

(30) Foreign Application Priority Data
Nov. 12, 2002 (FR) 02/14115

(51) Int. Cl. A61K 7/06; A61K 7/11

(52) U.S. Cl. 424/401; 424/70.13

(57) ABSTRACT

Greasy/shiny skin is cosmetically treated or matted by topically applying thereon a composition which contains a thus effective amount of cellulose microbeads formulated into a topically applicable, physiologically acceptable medium therefor, at least 90% of which cellulose microbeads, in numerical terms, having a diameter of less than or equal to 15 μm.

### CROSS-REFERENCE TO PRIORITY/PCT/PROVISIONAL APPLICATIONS

This application claims priority under 35 U.S.C. §119 of FR 02/14115, filed Nov. 12, 2002, and of provisional application Ser. No. 60/429,342, filed Nov. 27, 2002, and is a continuation of PCT/EP 2003/011646, filed Oct. 20, 2003 and designating the United States (published in the English language on May 27, 2004 as WO 2004/043329 A2); each hereby expressly incorporated by reference and each assigned to the assignee hereof.

### BACKGROUND OF THE INVENTION

1. Technical Field of the Invention

The present invention relates to the cosmetic formulation of cellulose microbeads, at least 90% of which, in numerical terms, have a diameter of less than or equals to 15 μm into compositions that are suitable for topical application onto the skin, as a matting agent.

The invention also relates to a cosmetic regime or regimen for treating greasy skin, comprising the topical application onto the skin of a composition containing cellulose microbeads formulated into a physiologically acceptable medium, at least 90% of which, in numerical terms, have a diameter of less than or equal to 15 μm.

2. Description of Background and/or Related and/or Prior Art

Shiny skin, which is often associated with a substantial secretion of sebum, is a problem that particularly affects adolescents, but may also be manifested in adulthood, due especially to the effect of an overproduction of androgens. It may also be associated with sweat resulting from physical activity or climatic conditions. However, shiny skin is considered as unattractive, all the more so since it often entails poorer staying power of makeup, which has a tendency to degrade visually in the course of the day.

To absorb the sebum and the excess oil of the composition that is not absorbed by the skin, use is conventionally made of powders of natural or synthetic origin, among which mention may be made especially of fillers such as talc, starch, mica, silica, nylon powders, polyethylene powders, poly-β-alanine and poly-methyl (meth)acrylate powders. Fillers of this type have the drawback of giving the skin an unnatural powdery appearance, which may even accentuate skin defects: Furthermore, the compositions containing these are generally dehydrating in the long term and their effect is not very long-lasting.

In addition, it has been proposed by the assignee hereof to use as matting agents vinylpyrrolidone/1-triacontene copolymers (FR-2,820,972) or melamine-formaldehyde or urea-formaldehyde resin particles (FR-2,792,642), styrene-acrylic resin particles (FR-2,801,215), polytetrafluoroethylene resin particles (FR-2,820,977) or colloidal dispersions of mineral particles, in particular of silica, which may be prepared via a sol-gel process (EP-0,682,939).

Although these matting agents have advantageous properties, need continues to exist for matting agents that have both good compatibility with the skin—and in particular that are of natural origin—and good sensory properties when applied.

### SUMMARY OF THE INVENTION

It has now surprisingly been determined that cellulose microbeads of judicious size makes it possible to formulate products for greasy skin that have good matting power, while at the same time affording good physiological compatibility, on account of the natural origin, and good cosmetic properties, in the sense that these products do not dry out the skin.

These cellulose microbeads, and the process for preparing them, have been described in the publication by ISHIHARA S. et al., Novel coloured complex cellulose beads, 22^{nd} Congress of the IFSCC, Edinburgh 2002. It is suggested to use them in cosmetics for the formulation of products with a soft feel and good moisturizing power on account of their water-absorbing capacity. These cellulose microbeads are also advantageous for absorbing oil, which makes it possible to envisage their use for improving the staying power of makeup products.

However, it is not suggested in the said document that these cellulose microbeads can have an instantaneous matting effect, i.e., give the skin a matt appearance immediately on application, and that they may thus be of interest precisely in caring for greasy skin.

Thus, it has now been demonstrated that certain cellulose microbeads, which are precisely such that at least 90%, in numerical terms, have a diameter of less than or equal to 15 μm, have the abovementioned matting properties.

Accordingly, the present invention features a cosmetic process for treating greasy skin, comprising the topical application to the skin of a composition containing cellulose microbeads in a physiologically acceptable medium, at least 90% of which, in numerical terms, have a diameter of less than or equal to 15 μm.

### DETAILED DESCRIPTION OF BEST MODE AND SPECIFIC/PREFERRED EMBODIMENTS OF THE INVENTION

For the purposes of the present invention, the expression “cosmetic treatment of greasy skin” means the topical application of skincare products, intended not only to instantaneously reduce the shininess of the skin, but also to make it healthy by providing it with various active agents intended to combat its imperfections, such as antibacterial or keratolytic agents.

For the purposes of the present invention, the term “microbeads” means solid or porous particles, i.e., microparticles rather than microcapsules, having a circularity parameter of at least 0.95. The circularity parameter is defined as the ratio of the circumference of a disc having the same area as the particle to the perimeter of the particle. A value of 1 characterizes particles that are perfectly spherical.

The microbeads according to the invention are such that at least 90%, in numerical terms, have a diameter of less than or equal to 15 μm. They preferably have a number-average diameter of not more than 10 μm, preferably ranging from 0.1 to 10 μm and better still from 0.5 to 10 μm.

The size distribution at the cellulose microbeads used according to the invention may be measured using an FPIA 2100 image analysis granulometer from the company Malvern.

These microbeads may be prepared especially as described in the abovementioned Ishihara publication, i.e., according to a process comprising the successive steps of mixing an aqueous solution of sodium polyacrylate and of viscose, of stirring the mixture, of heating to 80° C., of filtering, of acidic hydrolysis and of washing with water. They are moreover commercially available in the form of powder from the company Daito under the commercial reference Cellulo Beads D-5® or, better still, from the company LCW under the trade name Covabeads CLO®.

The present invention also features the cosmetic formulation of cellulose microbeads, at least 90% of which, in numerical terms, have a diameter of less than or equal to 15 μm, into compositions suitable for topical application to the skin, as a matting agent.

For the purposes of the present invention, the term “matting agent” means a raw material capable of instantaneously (immediately on application) reducing the shininess of the skin, i.e., the ratio R of the specular reflection to the diffuse reflection, as measured on a gonioreflectometer as described in Example 2 below.

The compositions according to the invention are suitable for topical application to the skin and thus generally comprise a physiologically acceptable medium, i.e., a medium that is compatible with the skin and/or its integuments. Thus, the compositions according to the invention preferably have a pH of less than 7 and better still ranging from 5 to 6.

The amount of cellulose microbeads present in the composition may vary within a wide range depending on the desired effect. Its very good sensory characteristics allow it to be formulated in fairly high contents, while at the same time maintaining a soft and highly cosmetic texture for the composition containing them. By way of example, these particles may represent from 0.1 % to 25% by weight, preferably from 0.5% to 20% by weight and better still from 5% to 15% by weight relative to the total weight of the composition.

The compositions according to the invention may be in any presentation form conventionally used for topical application and especially in the form of aqueous gels or aqueous or aqueous-alcoholic solutions. By adding a fatty or oily phase, they may also be in the form of dispersions or emulsions of liquid or semi-liquid consistency of the milk type, obtained by dispersing a fatty phase in an aqueous phase (O/W) or conversely (W/O), or of suspensions or emulsions of soft, semi-solid or solid consistency of the cream or gel type, or alternatively multiple emulsions (W/O/W or O/W/O emulsions), microemulsions, vesicular dispersions of ionic and/or nonionic type, or wax/aqueous phase dispersions. These compositions are prepared according to the usual methods.

According to one preferred embodiment of the invention, the composition is in the form of an oil-in-water emulsion.

In this case, the proportion of the oily phase of the emulsion may range, for example, from 1% to 40% by weight relative to the total weight of the composition. The oils, emulsifiers and co-emulsifiers used in the composition in emulsion form are selected from among those conventionally used in cosmetics or dermatology. The emulsifier and the co-emulsifier are generally present in the composition in a proportion ranging from 0.3% to 30% by weight and preferably from 0.5% to 20% by weight relative to the total weight of the composition.

When it is in the form of an O/W emulsion, the composition according to the invention may contain, as surfactants, at least one compound selected from among: polyol esters of fatty acids with a saturated or unsaturated chain containing, for example, from 8 to 24 carbon atoms and better still from 12 to 22 carbon atoms, and the oxyalkylenated derivatives thereof, i.e., derivatives comprising oxyethylene and/or oxypropylene units, such as glyceryl esters of C₈-C₂₄ fatty acids, and the oxyalkylenated derivatives thereof, sorbitol esters of C₈-C₂₄ fatty acids, and the oxyalkylenated derivatives thereof, sugar (sucrose, glucose or alkylglucose) esters of C₈-C₂₄ fatty acids, and the oxyalkylenated derivatives thereof; polyethylene glycol esters of C₈-C₂₄ fatty acids, and the oxyalkylenated derivatives thereof; polyalkylene glycol ethers of C₈-C₂₄ fatty alcohols; sugar ethers of C₈-C₂₄ fatty alcohols, and mixtures thereof.

As a variant, the compositions according to the invention in emulsion form may contain an ionic amphiphilic polymer and may be free of emulsifier.

The compositions according to the invention may also contain the adjuvants that are common in cosmetics and dermatology, such as hydrophilic or lipophilic gelling agents, active agents, preservatives, solvents, fragrances, fillers, pigments, odor absorbers and dyestuffs. The amounts of these various adjuvants are those that are conventionally used in the fields under consideration, for example from 0.01% to 20% of the total weight of the composition. Depending on their nature, these adjuvants may be introduced into the fatty phase or into the aqueous phase. These adjuvants and the concentrations thereof should be such that the do not adversely affect the advantageous properties of the cellulose microbeads according to the invention.

As active agents, the compositions according to the invention will preferably contain at least one active agent selected from among retinoids and in particular retinol; zinc salts such as zinc gluconate; an extract of Laminaria saccharina; an extract of wild yam; triclosan; phenoxyethanol; octoxyglycerol; octanoylglycine; an extract of clove; caprylyl glycol; azelaic acid; α-hydroxy acids such as lactic acid or glycolic acid; β-hydroxy acids, in particular salicylic acid and its derivatives such as 5-n-octanoylsalicylic acid; ursolic acid; panthenol; niacinamide; octopirox.

Examples of fillers that may be mentioned include polyamide (Nylon) particles and especially those sold under the name Orgasol by the company Atochem; polyethylene powders; microspheres based on acrylic copolymers, such as those made of ethylene glycol dimethacrylate/lauryl methacrylate copolymer sold by the company Dow Corning under the name Polytrap; polymethyl methacrylate microspheres, sold under the name Microsphere M-100 by the company Matsumoto or under the name Covabead LH85 by the company Wackherr; ethylene-acrylate copolymer powders, for instance the products sold under the name Flobeads by the company Sumitomo Seika Chemicals; expanded powders such as hollow microspheres, and especially the microspheres formed from a terpolymer of vinylidene chloride, of acrylonitrile and of methacrylate, sold under the name Expancel by the company Kemanord Plast under the references 551 DE 12 (particle size of about 12 μm and density of 40 kg/m³), 551 DE 20 (particle size of about 30 μm and density of 65 kg/m³), 551 DE 50 (particle size of about 40 μm), or the microspheres sold under the name Micropearl F 80 ED by the company Matsumoto; powders of natural organic materials such as starch powders, especially of crosslinked or non-crosslinked maize, wheat or rice starch, such as the starch powders crosslinked with octenylsuccinic anhydride, sold under the name Dry-Flo by the company National Starch; polyamide fibers; silicone resin microbeads such as those sold under the name Tospearl by the company Toshiba Silicone, especially Tospearl 240; silica; metal oxides such as titanium dioxide, zinc oxide or alumina; mica; talc; sericite; boron nitride; clays; and mixtures thereof.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Matting Cream (O/W Emulsion):

Oily Phase:

| | |
|---|---|
| cyclohexadimethylsiloxane | 10.00% |
| Stearyl alcohol | 1.00% |
| Glyceryl stearate and polyethylene glycol | 2.00% |
| stearate (100 EO) | |
| Dimyristyl tartrate/cetearyl | 1.50% |
| alcohol/oxyethylenated (25 FO) | |
| oxypropylenated (25 PO) lauryl alcohol/oxy- | |
| ethylenated (7 EO) C₁₂-C₁₅ alcohols | |
| (Cosmacol PSE from the company Enichem) | |

Aqueous Phase:

| | |
|---|---|
| Xanthan gum | 0.20% |
| Crosslinked poly(2-acrylamido-2-methyl- | 0.40% |
| propanesulfonic acid) sold by the company | |
| Hoechst under the name Hostacerin AMPS | |
| Sodium hydroxide | 0.01% |
| Glycerol | 5.00% |
| Modified starch sold by the company | 3.00% |
| National Starch under the name Dry-Flo | |
| Cellulose microbeads sold by the company | 3.00% |
| Daito under the name Cellulo Beads D-5 | |
| Preservatives | 0.70% |
| Water | qs 100.00% |

Procedure:

The above composition was prepared in the following manner. The aqueous and oily phases were prepared separately by mixing their constituents and then heating to 75° C. The oily phase was then introduced slowly into the aqueous phase with stirring using a Moritz blender at a speed of 4000 rpm for 20 minutes.

### Example 2

### Demonstration of the Matting Effect:

Protocol:

Three compositions A to C were tested, whereby:
composition A corresponded to that of Example 1,
composition B was identical to that of Example 1, except that the cellulose microbeads were those sold by the company Daito under the name Cellulo Beads D-10,
composition C was identical to that of Example 1, except that the cellulose microbeads were replaced with the same amount of silica microbeads sold by the company Miyoshi under the name SB1SO (number-average diameter: 3-15 μm).

The Cellulo Beads D-5 cellulose microbeads have a number-average diameter of 10 μm, and are such that 90%, in numerical terms, of the particles have a diameter of less than 15 μm, 50%, in numerical terms; of the particles having a diameter of less than 9 μm.

The Cellulo Beads D-10 cellulose microbeads have a number-average diameter of 13 μm, and are such that 90%, in numerical terms, of the particles have a diameter of less than 23 μm, 50%, in numerical terms, of the particles having a diameter of less than 14 μm.

The principle of the method entails measuring, using a gonioreflectometer, the specular reflection and the diffuse reflection of a deposit of each composition to be evaluated, spread beforehand at room temperature onto a rubber support at a rate of 2 mg/cm². The deposits are then dried at room temperature for 30 minutes.

The matting effect is then evaluated from the ratio R of the specular reflection (measured at an angle of 30°) and diffuse reflection (measured at an angle of 0°).

The lower this ratio, the higher the matting effect. In general, a ratio R of greater than 2 is considered as reflecting an absence of improvement of the appearance of the skin.

Results:

The results obtained for the three test compositions are reported in the following table:

TABLE

| Composition | R |
|---|---|
| A | 1.77 ± 0.04 |
| B | 2.32 ± 0.17 |
| C | 1.91 ± 0.09 |

It is thus seen from the above table that the cellulose microbeads in accordance with the invention have a higher matting power than the cellulose microbeads of larger diameter and than the silica microbeads. It is also seen that composition B containing cellulose microbeads, at least 90% of which have a diameter of less than 15 μm will have no effect on the matt appearance and the uniformity of the complexion.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A cosmetic regime or regimen for the treatment of greasy skin, comprising topically applying onto the greasy skin of an individual in need of such treatment, a composition which comprises a thus effective amount of cellulose microbeads formulated into a topically applicable, physiologically acceptable medium therefor, at least 90% of which cellulose microbeads, in numerical terms, having a diameter of less than or equal to 15 μm.

2. The cosmetic regime or regimen as defined by claim 1, said cellulose microbeads having a number-average diameter ranging from 0.5 to 10 μm.

3. The cosmetic regime or regimen as defined by claim 1, said cellulose microbeads have a circularity parameter of at least 0.95.

4. The cosmetic regime or regimen as defined by claim 1, said composition comprising from 5% to 15% by weight of said cellulose microbeads.

5. The cosmetic regime or regimen as defined by claim 1, said composition comprising an oil-in-water emulsion.

6. The cosmetic regime or regimen as defined by claim 1, said composition having a pH of less than 7.

7. The cosmetic regime or regimen as defined by claim 1, said composition having a pH ranging from 5 to 6.

8. A cosmetic regime or regimen for the treatment of shiny skin, comprising topically applying onto the shiny skin of an individual in need of such treatment, a composition which comprises a thus effective amount of cellulose microbeads formulated into a topically applicable, physiologically acceptable medium therefor, at least 90% of which cellulose microbeads, in numerical terms, having a diameter of less than or equal to 15 μm.

9. A cosmetic regime or regimen for the matting of human skin, comprising topically applying onto the skin of an individual in need of such treatment, a composition which comprises a thus effective amount of cellulose microbeads formulated into a topically applicable, physiologically acceptable medium therefor, at least 90% of which cellulose microbeads, in numerical terms, having a diameter of less than or equal to 15 μm.

10. The cosmetic regime or regimen as defined by claim 1, said cellulose microbeads having been prepared by successively mixing an aqueous solution of sodium polyacrylate and of viscose, stirring the mixture, heating same to 80° C., filtering same, then conducting acidic hydrolysis thereof and lastly washing same with water.

11. A topically applicable cosmetic composition suited for the matting or treatment of greasy/shiny skin, having a pH of less than 7, and comprising a thus effective amount of cellulose microbeads formulated into a topically applicable, physiologically acceptable medium therefor, at least 90% of which cellulose microbeads, in numerical terms, having a diameter of less than or equal to 15 μm.

12. The cosmetic composition as defined by claim 11, having a pH ranging from 5 to 6.

13. The cosmetic composition as defined by claim 11, comprising from 5% to 15% by weight of said cellulose microbeads.

14. The cosmetic composition as defined by claim 11, comprising an oil-in-water emulsion.
